(19) United States
(12) Patent Application Publication
(10) Pub. No.: US 2015/0038955 A1
(43) Pub. Date: Feb. 5, 2015

(54) SKIN CONTACT DETECTING DEVICE FOR A DEVICE TO BE SECURED

(71) Applicant: Pantec Biosolutions AG, Ruggell (LI)

(72) Inventors: Thomas Bragagna, Mauren (LI); Clemens Hagen, Maeder (AT); Arne Heinrich, Feldkirch (AT); Peter Krammer, Buchs (CH); Stefan Summer, Dafins (AT)

(73) Assignee: PANTEC BIOSOLUTIONS AG, Ruggell (LI)

(21) Appl. No.: 14/358,407

(22) PCT Filed: Nov. 16, 2012
(86) PCT No.: PCT/EP2012/072860

§ 371 Date: Sep. 12, 2014

Related U.S. Application Data
(60) Provisional application No. 61/795,454, filed on Oct. 17, 2012.

(30) Foreign Application Priority Data
Nov. 16, 2011 (AT) A 1704/2011

(51) Int. Cl. A61B 18/20

(52) U.S. Cl. CPC A61B 18/203; A61B 2017/00066
USPC 606/12; 606/1

(57) ABSTRACT

The invention relates to a skin contact detecting device (1) for a device (24) to be secured, comprising a contact piece (2) with a bearing surface (3) and comprising a safety circuit (7) with a light source (6), a photodetector (9), and an analyzing circuit (17). The bearing surface (3) has a light outlet portion (4) and a light inlet portion (5) which is delimited by the light outlet portion. Furthermore, the light source (6) has a main beam direction (8) of the emitted electromagnetic radiation, said main beam direction being oriented in the direction of the light outlet portion (4). The photodetector (9) has a detecting region (10) for electromagnetic radiation, said detecting region being oriented in the direction of the light inlet portion (5). The light source (6) is designed to emit light of a first (13) and at least one second (14) wavelength, and the photodetector (9) is sensitive to the first (13) and the second (14) wavelength. The analyzing circuit (17) has a database (43) in which reference data records (41, 42) comprising reference data for detected first (39) and second (40) signals are stored. Furthermore, the analyzing circuit (17) has a comparator circuit (19), said comparator circuit (19) being designed to compare a detected first (39) and second (39) wavelength with the reference data records (41, 42) stored in the database (43) and to emit a secure operating state signal (22).

This application claims priority of the provisional U.S. patent application Ser. No. 61/795,454 filed on 17 Oct. 2012.

The invention relates to a skin contact detecting device.

For many devices a clearly defined working environment is crucially important to prevent persons performing a treatment and persons receiving a treatment being put at risk. For example, the operation of the device could involve the emission of an energy form that is potentially hazardous to health in order to achieve a desired effect on the skin surface or on biological tissue. The emitted energy can be for example high energy laser radiation, in particular laser radiation classified in laser class 4, which if it came into direct contact with an eye would cause irreversible damage to the latter. In addition, the uncontrolled impact of such radiation on the skin could result in severe damage to the skin tissue.

Until now it has been known that such systems can only be operated in specially protected environments, for example devices of this kind have to be set up and operated in an access-controlled room in which all persons had to wear protective glasses during the operation of the device and if necessary protective clothing. Furthermore, the operation and use of such a device was reserved for specifically trained specialists, which stands in the way of the widespread distribution of such devices; in particular it would be inconceivable to use such devices in the home or outside special facilities.

With regard to the hazards a classification was introduced specifically for laser systems (according to EN 60825-1), in which essentially only laser class 1 is approved for general use, as when using a laser class 1 device it is ensured that any potentially emitted laser radiation cannot cause damage to the eye or skin even in the case of direct impact on the latter.

A further aspect of the invention relates to devices or equipment which on initial operation can emit substances or particles that are hazardous to health. For example, this includes every type of explosive or chemically or biologically active device, in particular detonation devices and firearms as well as releasing devices for biological and/or chemical substances.

In both situations it is therefore crucially important that it is possible to ensure that the device to be secured is in direct contact with biological tissue, in particular skin contact is made, prior to the initial operation or during operation.

From the prior art safety systems are known which determine the electrical parameters of biological tissue and also establish whether there is skin contact. For example EP 1 898 825 A2 shows a device which determines the impedance of the biological tissue and also detects skin contact. Such systems have the disadvantage that they can be circumvented relatively easily, for example in that a resistance network is applied to the contact electrodes and thus biological tissue is simulated. For applications in the field of medicine electrical contact with a person has the further disadvantage that very high safety standards have to be met if a person comes into conductive contact with a device which is connected to the power supply network. In addition, such electrical contact would fail if a protective layer or cover layer were applied to the surface of the skin.

Furthermore, from the prior art force-using skin contact detecting devices are known in which a skin section has to press with a specific, mechanical force onto a contact piece, in order to put the device to be secured into operation. Such devices, for example EP 1 460 955 A1, are very easy to circumvent however, as an activating element can be created which charges the contact piece with the necessary force and thus simulates the skin contact and sets the device to be secured into operation incorrectly.

Furthermore, from U.S. Pat. No. 7,413,567 B2 a device is known which detects the reflection of various different emitted wavelengths and by comparison with previously determined reference values determines the application of the sensor on the skin. To establish the presence of skin the sensor is firstly placed on the skin and on confirmation the darkness value is determined which is used as a reference for further measurements. Afterwards when the sensor has been lifted light is emitted successively in at least three different wavelengths. By forming the difference between the reflected light and the darkness value a value field is determined which is checked against threshold values in order to thus determine the approach towards the skin. The disadvantage here is that first of all a darkness value has to be determined which provides an opportunity for manipulation. The darkness value is determined in this system by a user action; it is not possible for the sensor to determine with certainty the actual application onto the skin. Thus a residual brightness can also be determined as a darkness value by an imprecisely positioned sensor, so that the subsequent detection of an approach would identify the placing of the sensor on the skin too early.

A similar system is also known from U.S. Pat. No. 8,098,900 B2. The document discloses a device in which by means of light in two different wavelengths the presence of human skin is determined in the detecting area. Here too the sensor is placed on the skin to determine a reference value in order to then make the distinction.

From EP 2 054 193 B1 a safety device for a machine tool is also known in which a radiation unit emits light in at least two different wavelengths in a detecting area and by evaluating the reflected light determines whether there is human tissue or material, tools or objects in the detecting area and in the case of human tissue activates protection. Devices known from the prior art function either on the principle of contact (by force or electrically) or require a reference value to be determined or enable only a qualitative definition of the presence of skin. In any case by means of the known devices the skin recognition can be mostly manipulated very easily. Contacts can be fixed for example by holding means, an electrical impedance measurement can be manipulated by applying a substitute impedance. In the case of contactless systems by manipulating the definition of the reference value the safety function can be undermined. With regard to securing devices, which when handled incorrectly can cause harm to persons in the vicinity, it needs to be ensured that any manipulation attempt is recognized and prevented and that a supposedly safe operating state of the device to be secured is recognized and prevented.

The objective of the invention is thus to create a device which can determine unambiguously the application of a contact piece onto biological tissue, which can be used without electrical and/or mechanical contacting components and without calibration and by means of which it can be reliably established that the latter is biological tissue and the contact is made and secured. The intention is also to prevent any manipulation of the safety function caused by a deliberate or unconscious incorrect calibration. Furthermore, the device needs to be compact and consume as little electrical energy as possible with regard to battery driven or rechargeable battery driven devices.

The objective of the invention is achieved by a skin contact detecting device for a device to be secured, comprising a contact piece with an application surface, a safety circuit with a light source, a photodetector and an analyzing circuit, which analyzing circuit is connected to the photodetector. The application surface comprises a light outlet and a light inlet section delineated therefrom, the light source also has a main beam direction of the emitted electromagnetic radiation, wherein the main beam direction is aligned in the direction of the light outlet section. The photodetector has a detecting area for electromagnetic radiation, which detecting area is aligned in the direction of the light inlet section. The light source is designed for emitting light of a first and at least a second wavelength, and the photodetector is designed to be sensitive to the first and second wavelength and for converting a received first wavelength into a first detected signal and the received second wavelength into a second detected signal. The analyzing circuit comprises a database in which reference data records are saved with reference data for detected first and second signals. Furthermore, the analyzing circuit comprises a comparator circuit, which comparator circuit is designed for comparing a detected first and second wavelength with the reference data records saved in the database and for emitting a safe operating state signal.

In one development it can also be provided that the light source is also connected to the analyzing circuit in order to thus enable a correlation between the emitted and the detected light. In this way in an advantageous manner the interfering influences of environmental light are suppressed more effectively.

As the propagation conditions for light in the skin or in biological tissue depends heavily on the wavelength, by selecting two different wavelengths the placing of the contact piece on the skin can be reliably identified. For example one wavelength is selected so that the latter is only slightly weakened by the tissue whilst the other wavelength is strongly damped by the tissue. In the following description the second wavelength is defined as the one which is strongly weakened by the tissue. The application can be recognized in that after the approach of the contact piece to the skin surface and mostly an increase in the detected intensities of the two wavelengths, on application the detected intensity of the second wavelength drops significantly, whereas the intensity of the first wavelength only falls slightly. Depending on the absolute value of the second wavelength, the latter can be completely absorbed by the tissue, so that no intensity is detected for the second wavelength.

If during the safe operating state the present device is lifted from the skin, even only very slightly, there will be a change in the detected signals, whereby the safe operating state signal is deactivated or is no longer provided and the device to be secured can be deactivated straight away.

In the description the terms skin, skin surface and biological tissue are used synonymously. For the description of detailed features suitable terms are used.

Furthermore, the term safety function or variations thereof is defined to mean that the application of the contact piece on the skin is ensured.

In one development the first wavelength is smaller than 470 nm, in particular 450 nm. In particular the first wavelength lies in the range of visible blue up to the beginning of UV. For this wavelength range or for this wavelength biological tissue has a very high damping effect, so that light is absorbed by the tissue after only a very short distance. The lower the amount of light with the first wavelength reaching the light inlet section after placing the present device on the skin surface, the greater the change in the detected signals and the greater the clarity of recognition of the application. The wavelength also lies in a range in which there is a smaller proportion of natural light. In this way it is not possible to circumvent the safety function by arranging a light source next to the present device. Reference is made to the corresponding specialist literature with regard to the propagation conditions of different wavelengths in biological tissue. A wavelength of 450 nm is absorbed very efficiently by the melanocytes in the layer between the epidermis and stratum corneum (basal layer) and is used by the body for protection against damaging UV-radiation. The tanning action of the skin when sunbathing is based on this. Fitzpatrick classified six skin types, from type I (Celtic type, very light skin color) to type VI (dark brown to black skin); the darker the skin the greater the absorption.

According to one development the second wavelength is in a range of 610 nm to 660 nm, in particular 635 nm. This wavelength range or this wavelength has the advantage that biological tissue here has a low damping factor. Light that is directed into the biological tissue spreads out in the latter so that a suitably large application area can be secured.

According to one development the contact piece has a connecting surface lying opposite the contact surface, wherein the connecting surface is designed for the arrangement of the skin contact detecting device on the device to be secured. By means of this development a device is created which in a self-contained manner ensures the correct placing of the contact piece onto the biological tissue and thus can be flange-mounted on a plurality of devices to be secured. The connecting surface can also comprise electrical contact surfaces in order to supply the present device with electrical energy and in order to transmit the safe operating state signal to the device to be secured.

According to one development the light outlet section is connected via a first breakthrough in the contact piece and/or the light inlet section is connected via a second breakthrough in the contact piece to the connecting surface. In this way a configuration is created in which the contact piece only ensures the throughput of light from the light source to the biological tissue and from the latter back to the photodetector. This has the advantage that the contact piece can be in the form of a disposable part and thus also satisfies the high demands of the medical field.

One development, according to which the second breakthrough is formed by a light conductor, has the advantage that only a small and well-delineated section of the skin surface is located in the light inlet section. By means of the configuration of the end surfaces of the light conductor in the light inlet section the coupling condition can be determined very precisely so that any manipulation of the skin contact recognition is made more difficult or impossible by arranging a light source in the surrounding area of the contact piece. Light which reaches the light inlet section from a lateral direction would not be coupled into the light conductor. The light conductor can be made for example from synthetic fiber, preferably a PMMA fiber with a coating of fluorinated polymer with an NA of 0.55 and an aperture angle of 55°. Further possible embodiments are to be explored with respect to their wave-conducting properties in the required wavelength ranges.

In order to prevent any manipulation of the safety circuit according to one development the safety circuit is arranged in the device to be secured. In this way it can be ensured that none of the components ensuring the protective function are exposed to an attempt to manipulate them. This is an advantage in particular with respect to the long-term provision of the securing function. As the contact piece comes into contact with biological tissue this configuration is also advantageous as thus the contact piece can be subjected to a cleaning or sterilization process without putting the components of the safety circuit at risk in such a process.

By placing the contact piece on the skin surface the latter may be deformed, in particular in the areas of the light inlet or light outlet section. The device to be secured can comprise for example an optically functioning element which is dependent on a defined distance between the element and the skin surface. Therefore, one development is advantageous in which a spacing device is arranged in the light outlet section. Thus the deformation of the skin surface can be compensated or a defined curvature of the skin surface can be predetermined.

One development, according to which the light source is formed by at least one light-emitting diode or at least one laser diode, has the advantage that the wavelength of the emitted light can be adjusted very effectively and very precisely. In this way the wavelength can be adjusted effectively on the one hand to the damping level of the biological tissue. On the other hand the photodetector is sensitive to the emitted wavelength so that thus a pairing can be created which makes manipulation much more difficult. In particular in this way a manipulation attempt can be prevented with a strong, broadband light source, which is arranged in the vicinity of the contact piece or possibly recognized as such. A configuration of the light source as a laser diode has the advantage that a laser diode on the one hand emits a very narrow band light spectrum and in addition emits a very directed light beam, whereby a very good local resolution of the skin detection can be achieved. Particularly for the first wavelength light-emitting diodes are restricted in that they can provide only a very low brightness for blue and shorter wave light. Here laser diodes have the advantage that on the one hand components are available for shortwave blue and in addition can provide a high level of brightness. To be able to illuminate a larger surface area the beam of a laser diode has to be widened accordingly.

According to one development the light source is arranged in the light outlet section which has the advantage that in this way there is a very good coupling of the light emitted by the light source into the biological tissue.

According to one advantageous development in the application surface at least two contact electrodes are arranged which are connected to the analyzing circuit. The contact with biological tissue can also be performed by measuring electrical parameters for example by an impedance measurement or by determining dielectrical displacement currents.

In the medical field for network-connected electrical devices, which for example have a direct electrically conducting contact with a patient via sensors, there are very strict safety requirements with respect to the electrical insulation from the supply network. In one development according to which a galvanically isolated signal coupler is arranged between the contact electrodes and the analyzing circuit, a measurement of electrical parameters of the overlying tissue is possible without there being any direct contact of the network-operated device with the skin of a patient.

A further way of recognizing the correct application of the contact piece on the skin surface is obtained according to one development in which the light outlet section and/or the light inlet section is connected to a device for generating a differential pressure relative to the environment. On applying the contact piece the light outlet section and/or the light inlet section form together with the skin surface mostly an essentially airtight sealed space or a space with a clear pressure difference from the ambient pressure. Thus by analyzing the pressure conditions with a pressure detecting module of the analyzing circuit the application of the contact piece can be recognized in that a stable differential pressure is set and thus the safe operating state signal can be emitted. Any lifting of the contact piece can be recognized similarly by a change in the previously determined differential pressure. As no stable differential pressure is formed in the case of an incorrect application of the contact piece and the device will require increased effort to generate a differential pressure, this development enables a fine resolution detection of the correct application.

According to one development the photodetector is sensitive at least to a third wavelength, wherein said wavelength is selected such that it occurs in typical ambient light in a sufficient intensity. Furthermore, it should be able to be damped sufficiently by the tissue so that it is not possible during correct usage for said wavelength to be detected by the photodetector. As the use of the device to be secured does not usually take place in the dark the area of use is generally brightly illuminated. If the photodetector can detect at least one third wavelength, the lifting of the device can also be recognized in that suddenly components of the ambient light reach the photodetector.

A photodetector has a certain spectral range of sensitivity determined by the technology. As also in ambient light spectral components in the region of the first and second emitted wavelength can occur, it is possible that the latter could be detected incorrectly as originating from the light source. According to one development an optical filter element is arranged in the detecting area of the photodetector or in the photodetector. The filter element can now be configured such that it has a very sharp-edged passage characteristics, which can be adjusted in particular to the first and second emitted wavelength. Thus components of the ambient light and lighting bodies arranged with manipulative intent can be filtered out so that only the reflected or scattered first and second wavelength is detected. The filter element can now be arranged for example in the light inlet section. It is also possible however to arrange the filter element directly in the photodetector, for example as a filter layer on the photosensitive element.

According to one development the comparator circuit is designed to be state dependent, in particular as a state machine. In this way a sequence of the change of the detected signal can be determined which must occur in order to recognize a safe operating state. In particular, as the name implies, operation is performed in states which have to adopt an input variable in order to satisfy the saved sequence. In this case absolute values are not of interest, as long as the desired trend is maintained. As the detected signals clearly differ in intensity according to the skin type the main intensity sequence during the approach will be essentially similar for all skin types; with a state-dependent comparison the variability of the individual sequence can be managed.

According to one development the reference data of the reference data records has a hierarchy. By means of this development the dependency of the pattern of the detected signals can be ensured. In particular, the absolute intensity values of the detected signals are dependent on the skin type. As the reference data was determined by series of measurements on different skin types and saved as absolute values in the data records, during a comparison it is possible to determine for example which skin type is being approached.

According to one development the reference data of the reference data records is saved as pairs of range details. During preliminary analyses of a plurality of persons, divided into the main skin types according to Fitzpatrick, the reflection behavior of the skin was determined with regard to the first and second wavelength. This division into skin types cannot be delineated strictly, as on the one hand the skin has no quantized states and on the other hand the reflection behavior within a skin type depends heavily on the actual condition, such as for example on tanning. Thus the determined reference data will also include a certain amount of variation—as it is essentially a random process the distribution is a Gaussian distribution. Depending on the distance from the skin surface and skin type a value pair or a value field pair is saved, which for the two wavelengths gives an average value and a fluctuation margin. During the comparison it is possible to establish rapidly and reliably whether the detected first and second signal can be assigned to such a saved pairing. If an allocation is not possible, even after multiple repetitions, the detected first and second signal were very likely not reflected by the skin or there has been a manipulation attempt.

According to one development the reference data of the reference data records can have variation ranges. Unlike the previously mentioned range details concerning the fluctuation margin of the reference data, in this development a fuzziness is obtained regarding the full application of the contact piece. For example, when placing the contact piece on the skin over bony structures, for example on the face, it may occur that the contact piece is not applied completely so that a small amount of laser radiation can escape. On the basis of the optical/geometric conditions on or in the contact piece, the emerging laser radiation is scattered so much that it is not hazardous to health. The emerging laser radiation is thus in any case laser class I. As the intensity or amplitude of the detected first and second wavelength is strongly dependent on the spacing, thus for example over a saved variation range, relative to the amplitude conditions it is possible to define the distance from the application point. For example in this way a spacing of the contact piece from the skin surface of in the region of 0 to 2 mm can be considered safe, a range of 2 to 5 mm a warning range and everything above 5 mm results in a non-safe operating state signal.

In order to be able to take into consideration the unavoidable ambient light, according to one development the safety circuit has an ambient light sensor, which is connected to the analyzing circuit, in particular the latter is connected to the comparator circuit. A reliable recognition of the application onto the skin is only ensured when the intensity of the ambient light lies within predefined range. Light that is too bright would for example overpower the detected signals.

Likewise in order to take into account the ambient light according to one development the safety circuit has a pulse generator, which is designed for controlling the light source and is connected to the comparator circuit. By varying the intensity of the light emitted by the light source the latter can be extracted from the detected signals in order in this way to eliminate any influence on the ambient light.

According to one development the safety circuit comprises an additional light sensor which is aligned in the direction of the light outlet section and is connected to the analyzing circuit, in particular to the comparator circuit. The light of the light source is radiated over the light outlet section in the direction of the skin. As long as the contact piece does not lie on the skin or during the approach phase to the skin, hardly any or little light is reflected back because of reflection on the skin. The closer the contact piece comes to the skin, the more light is reflected by the skin, not only in the direction of the light inlet section and thus to the photodetector, but also back into the light outlet section. This light is detected, wherein the maximum reflection is achieved when the contact piece lies on the skin surface. As soon as the contact piece is lifted or tilted light will escape into the environment and thus the intensity of the signal detected by the further light sensor will fall. The signal provided by the additional light sensor can now be used as an additional safety criterion for recognizing the application on the skin and thus the safe operating state.

The objective of the invention is also achieved by a method for detecting the approach and the application of a device onto the skin surface. In this case a light source emits a first and second emitted wavelength via the light outlet section in the direction of the skin surface. The first and second wavelength reaching the photodetector via the light inlet section is detected by the photodetector and converted into a first and second detected signal proportional to the intensity. By means of the analyzing circuit for the first and second detected signal a first and second time signal pattern is formed, whereby by the comparator circuit of the analyzing circuit the first and second signal pattern are compared with the reference data records stored in the database. Furthermore, the analyzing circuit then emits a safe operating state signal when the first and second detected signal pattern coincides with reference data for first and second detected signal patterns.

According to one development the first and second signal patterns are determined by means of a smoothing function. As a plurality of environmental signals can overlayer the detected first and second signal and the approach to the skin need not necessarily be performed at constant speed, for example the user may tremble slightly, the overlayering may result in exceeding saved threshold values. By means of a smoothing process such, mostly very brief, fluctuations can be eliminated, and the determined signal pattern has no or only negligible artefacts. A non-conclusive list of such smoothing functions comprises for example a sliding linear average value, spline interpolation, Gaussian envelope curve, window function, standardization. Additional functions will be known to the person skilled in the art for eliminating the interfering portions of a signal with overlayered interference or not supplying such detected signals for further processing.

The application of the contact piece is characterized by a mostly very unambiguous signal constellation of the first and second detected signal, mostly the first detected signal disappears almost completely. During a comparison with strict boundaries on detecting a first detected signal a non-safe operating state signal is emitted and the device to be secured is deactivated. According to one development it is therefore the case that the comparator circuit performs a fuzzy comparison and in addition to the coincidence emits at least one second operating state signal, in particular a warning signal. In this way spacing ranges can be defined in which for example a warning signal is sent to the user, the device remains active however. Only at an even greater distance is the not-safe operating state signal emitted and the device deactivated. As already mentioned above with a very small deviation from complete application, for example in a range of 2 mm to 5 mm, only scattered or diffuse laser radiation can escape which cannot represent a hazard to health. The user should however be informed of this in order to align the contact device correctly to prevent switching off. If necessary, a timer can also be provided if necessary which is started on recognizing a warning state and the device deactivated if the warning state is maintained over too long a period. Preferably, the warning state is indicated to the user visually and/or acoustically.

In preliminary investigations which resulted in the present invention it has been shown that on approaching the skin, the levels of the first and second detected signals increase to a maximum and then drop accordingly. It has been deduced that this maximum for the present embodiment of the contact piece is about 10 mm from the skin surface. In another embodiment of the contact piece, particularly when changing the light outlet and inlet section, this value can of course change. It is important that this maximum is available and can be used as the marked reference point for determining the distance. Therefore, according to one development during the formation of the time signal patterns, a maximum value is found in order in this way to determine a known distance.

As interference can overlayer the detected first and second signal, which can have a direct influence on the formation of the signal patterns, according to one development on the formation of the time signal patterns a trend analysis is performed. For example by means of a trend analysis statistical anomalies can be evaluated correctly.

Thus according to one development the detected trends are compared with trends saved in the reference data records or with reference trends determined from the reference data records. This is another way to recognize the skin type and/or a correct approach or application.

An excellent condition for applying the contact piece onto the skin is characterized according to one development in that on applying the device or the contact piece on the skin almost no first signal is detected. Almost no signal means that the amplitude of the first detected signal is clearly below the amplitude of the second detected signal or the amplitude of the first detected signal is below the sensitivity threshold of the photodetector. As the first wavelength is short the latter is absorbed by the melanocytes in the skin, so that hardly any or no light emerges from the light outlet area via the skin into the light inlet area.

As a result of investigations the maximum value of the detected first and second signal is determined to be a distance of about 10 mm between the skin and present design of the contact piece. According to one development the determined signal patterns are interpolated linearly from the determined maximum value. By means of the interpolation an interpretation can be made of the detected first and second signal value of the distance between the contact piece and skin, in order thus to determine very precise spacing ranges, in which there is a safe operating state or a warning state. On the basis of the small distance a linear interpolation is permissible, however also an exponential interpolation could be used, for example a quadratic interpolation.

According to an advantageous development the light inlet section is charged with negative pressure or overpressure by a device for generating a differential pressures and the differential pressure is monitored by a pressure measuring device, and on exceeding a pressure threshold value of the differential pressure the safe operating state signal is emitted and on falling below a pressure threshold value of the differential pressure the non-safe operating state signal is emitted. In this way as an additional safety measure the tightness of the contact piece relative the environment is also used.

The invention is also achieved by a laser device of laser class 1, which comprises a laser source with a control circuit and a skin contact detecting device and wherein the control circuit ( ) of the laser source is only activated when there is a safe operating state signal. The laser source produces a radiation output that is greater than class 1, in particular class 4. By means of the configuration according to the claims it is ensured that a laser source hazardous to health is designed such that during operation no radiation can escape that is harmful to health and the laser device is thus in laser class 1.

For a better understanding of the invention the latter is explained in more detail with reference to the following Figures.

In a diagrammatic much simplified representation:

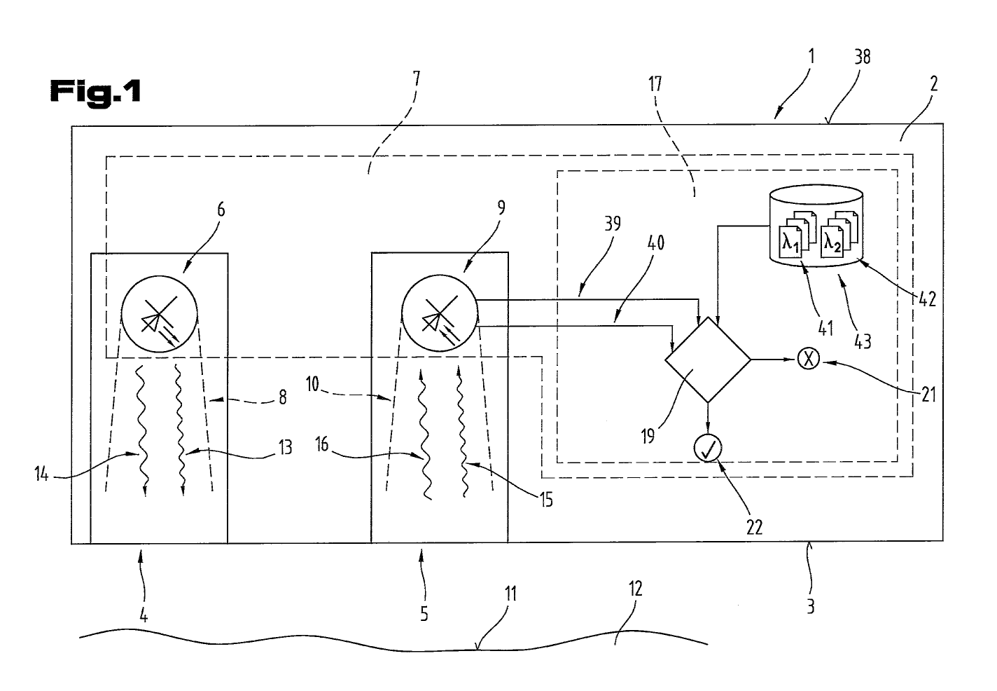

FIG. 1 is a block diagram of the present skin contact detecting device;

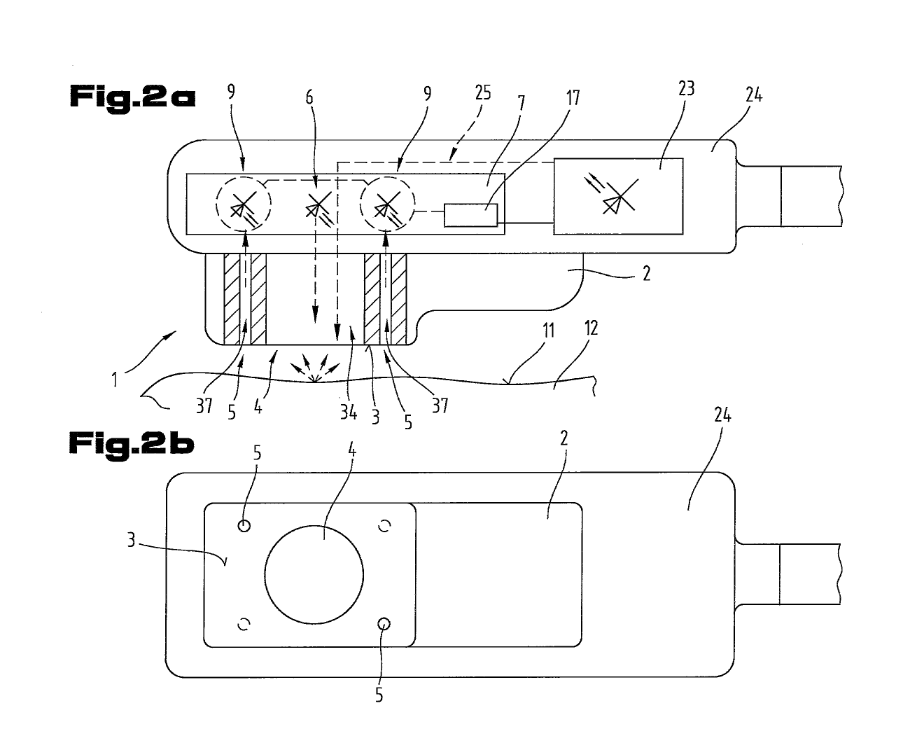

FIGS. 2 a) and b) a handpiece of an optically acting energy source with the present skin contact detecting device;

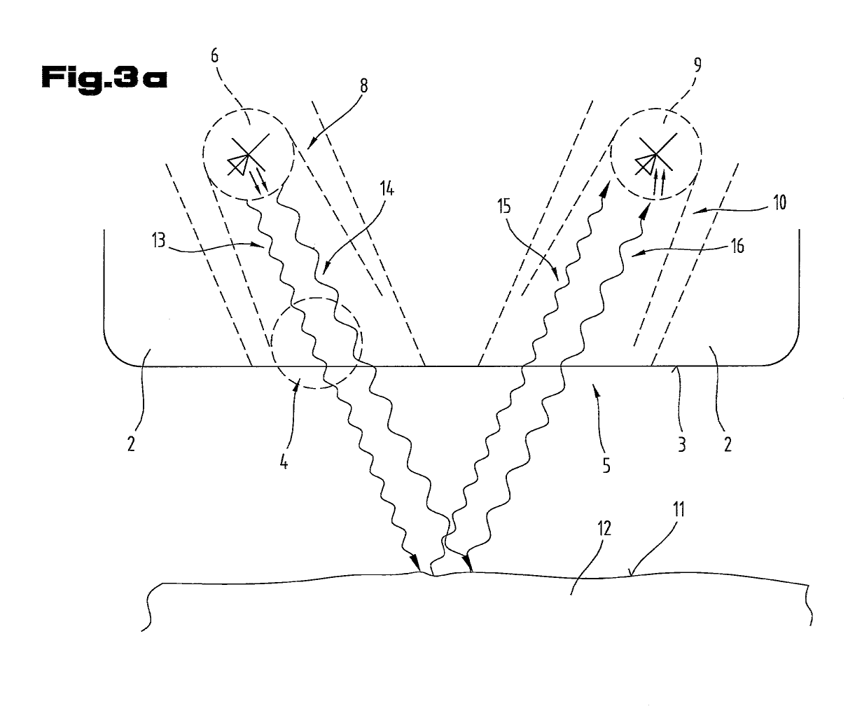

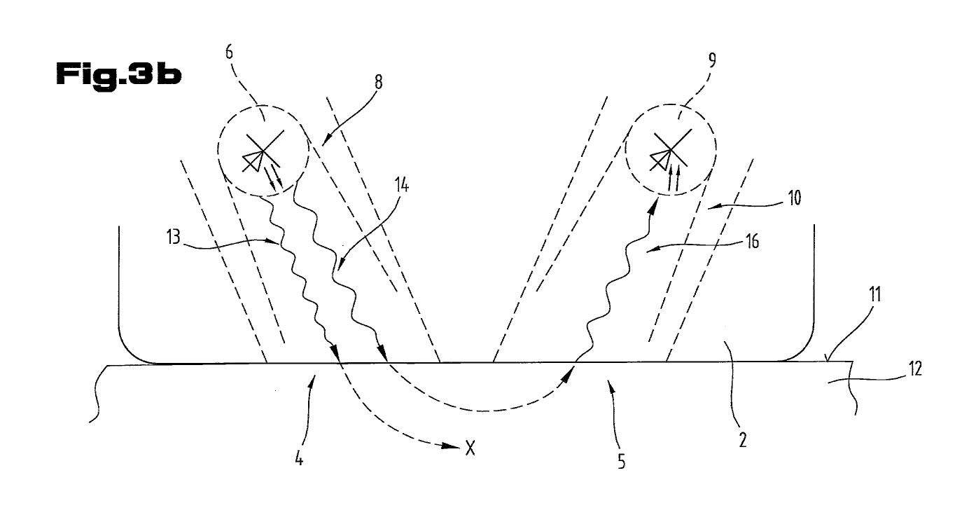

FIGS. 3 a) and b) the radiation conditions when moving the present skin contact detecting device towards biological tissue and placing it thereon;

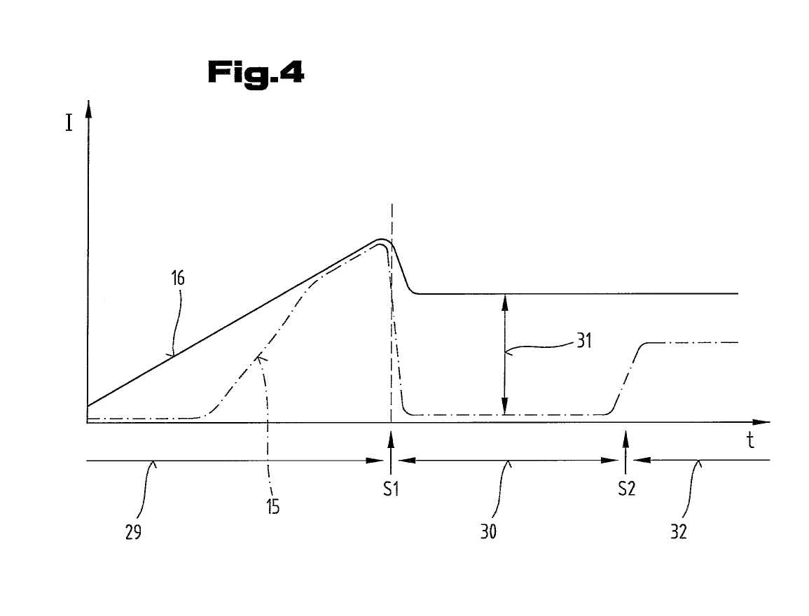

FIG. 4 a schematic pattern of the detected intensities of the two wavelengths;

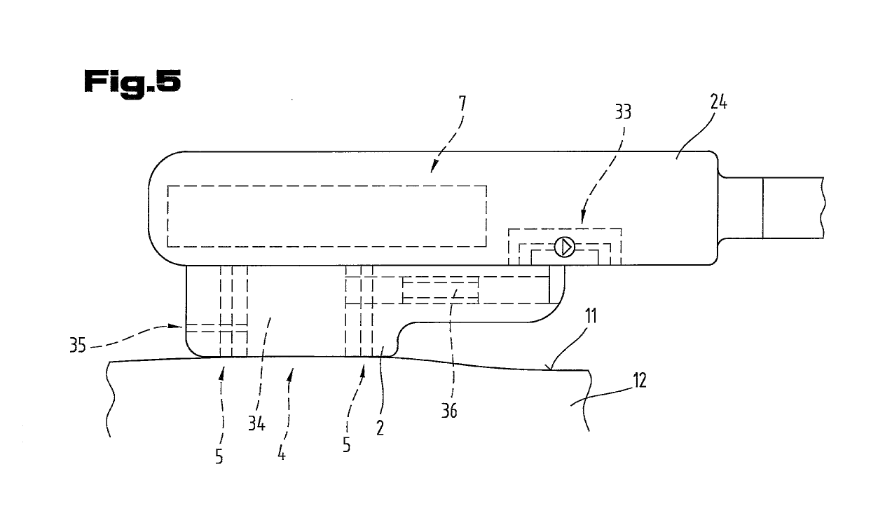

FIG. 5 a further possible embodiment of the present skin contact detecting device with a differential pressure device;

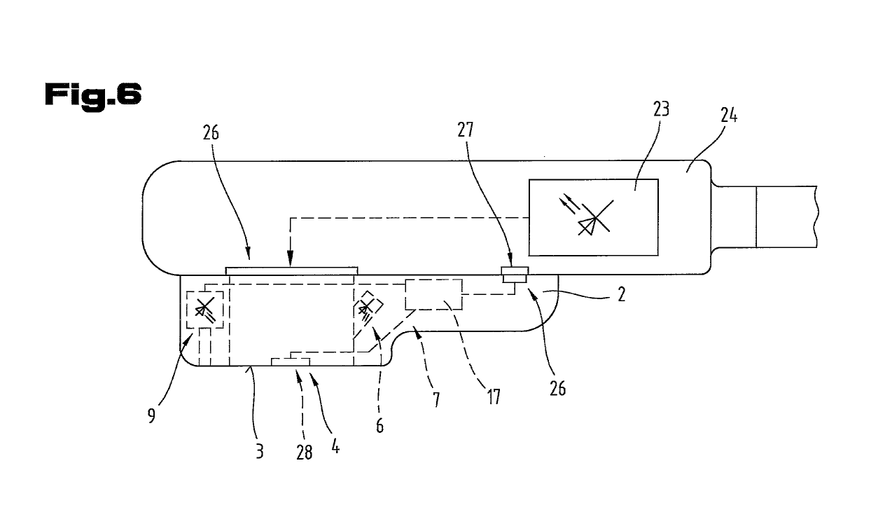

FIG. 6 a further possible embodiment of the present skin contact detecting device with a contact electrode;

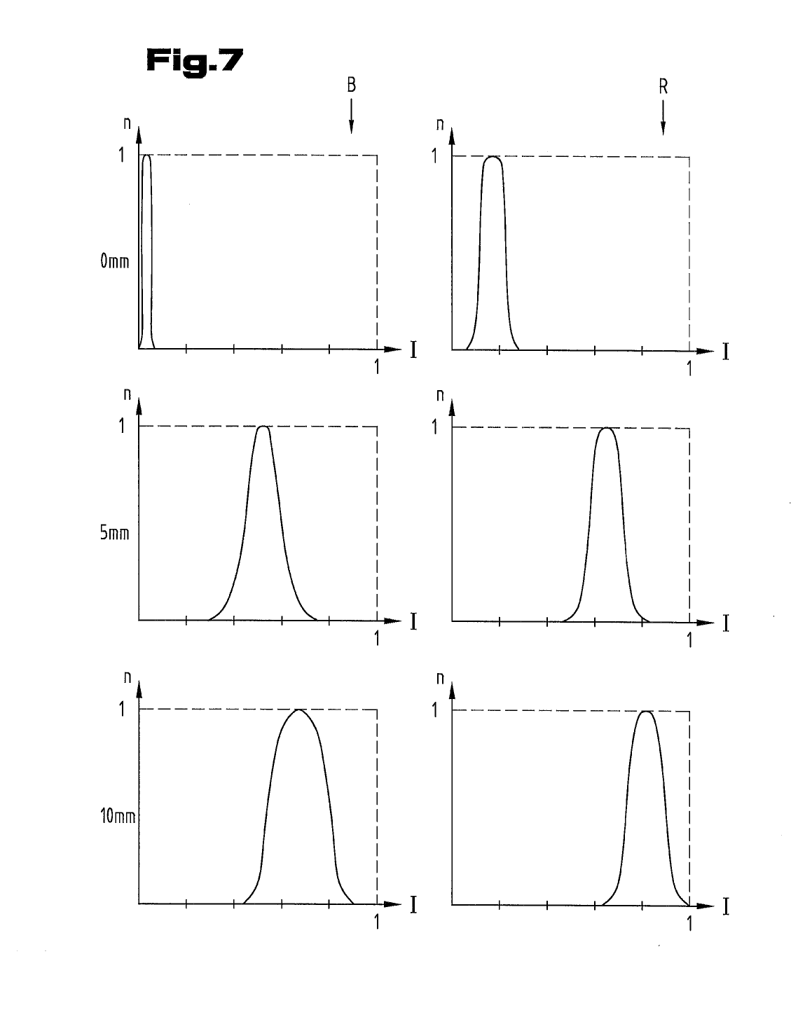

FIG. 7 an example of signal distribution when approaching the skin and placing thereon;

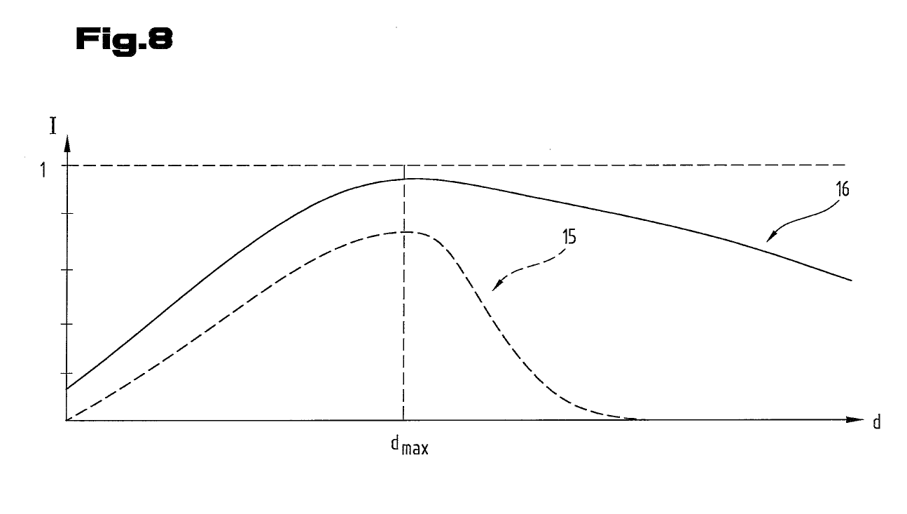

FIG. 8 an example of a signal pattern for distribution from FIG. 7.

FIG. 1 shows a schematic representation of the present skin contact detecting device 1, comprising a contact piece 2 with an application surface 3. The application surface 3 comprises a light outlet 4 and a light inlet section 5. A light source 6 of the safety circuit 7 is arranged with its main radiation direction 8 of the emitted electromagnetic radiation in the direction of the light outlet section 4. Furthermore, a photodetector 9 of the safety circuit 7 is arranged with its detecting area 10 in the direction of the light inlet section 5. The light inlet 4 and light outlet section 5 are arranged delineated from one another in the application surface 3 so that there is no direct radiation path of the light source 6 in the direction of the photodetector 9. Light from the light source 6 thus only reaches the light inlet section 5 via reflection on a surface 11 or by passing through tissue 12 and thus into the detecting area 10 of the photodetector 9.

Opposite the contact surface 3 the contact piece 2 comprises a connecting surface 38 by means of which the contact piece 2 can be connected to the device 24 to be secured. The connecting surface 38 is preferably configured such that a simple and reliable arrangement of the contact piece 2 on the device 24 is possible. A detachable arrangement is preferred, as according to one possible embodiment the contact piece 2 can be in contact with biological tissue and thus corresponding cleaning and sterilization has to be possible. This type of cleaning would probably damage the device 24 or reduce its lifetime, therefore it is an advantage if only the part in direct contact with the tissue is cleaned intensively. The connecting surface is preferably part of an aligning and locking device which engages in the part opposite the device 24 and arranges the contact piece 2 reliably on the device 24 to be secured.

The light source 6 is configured to emit electromagnetic radiation of at least two wavelengths in the optical range, in particular a shortwave, first emitted wavelength 13 and a second emitted wavelength 14 longer than the first emitted wavelength. The photodetector 9 is adjusted with regard to its sensitivity to the electromagnetic radiation emitted by the light source 6 to the first 13 and second wavelength 14, the electromagnetic radiation reaches the photodetector 9 as the first received wavelength 15 and second received wavelength 16.

By means of the photodetector 9 the received first 15 and second 16 wavelength is converted respectively into a signal 39, 40 proportional to the intensity and analyzed in an analyzing circuit 17. In particular, the two detected signals 39, 40 of a comparator circuit 19 are compared with reference data records for the first 41 and second 42 detected signals saved in a database 43. Depending on the comparison either a non-safe operating state signal 21 or a safe operating state signal 22 is emitted. In the reference data records 41, 42 reference data is saved for each of the two detected signals 39, 40. In particular, said reference data records were determined in series of measurements and saved in the database 43. In the series of measurements reference data was determined for different skin types, wherein the absolute level of the intensity of the received wavelengths 15, 16 and thereby the intensity of the detected signals 39, 40 depends heavily on the skin type. By using a database 43 with saved reference data records 41, 42 it possible that a reliable approach towards the skin or an application onto the skin is recognized without a previous calibration step being necessary. The prior art, for example U.S. Pat. No. 7,413,567 B2, discloses that prior to performing the actual positioning check the device has to be applied deliberately onto the tissue, in order to determine a reference point. By means of this calibration step however very simple manipulation is possible, as the device can thus be placed on any substrate and thus a completely false reference point is determined. With regard to safeguarding an optical high energy radiation source this kind of simple manipulation is a considerable disadvantage.

FIG. 2a shows a representation of the present skin contact detecting device 1 for safeguarding an optical high energy radiation source 23 which is arranged in a handpiece 24. In a preferred application the optical high energy radiation source 23 is a laser, in particular a laterally pumped solid body laser which emits optical high energy radiation 25 in the direction of a surface 11 to be treated. Preferably, the emission of the high energy radiation is performed via the light outlet section 4. The energy content of the emitted radiation 25 is thus so high that if radiation contacts the human eye the latter would be damaged immediately, in particular the radiation is assigned to class 4 according to EN 60 825-1. During the operation of such a device 24 without the present skin contact detecting device comprehensive protective measures are necessary in any case, in particular all of the persons involved need to wear protective glasses at least in the vicinity of the device 24. If this kind of costly protection is to be avoided it needs to be ensured that during the operation of the optical high energy radiation source 23 the maximum optical radiation energy can escape into the surrounding area of the device, which is assigned to class 1 and is thus not classified as hazardous to health. By means of the present skin contact detecting device 1 it is ensured in particular by means of the analyzing circuit 17 that the radiation source 23 can only be put into operation if by means of the safety circuit 7 or the analyzing circuit 17 the application of the contact piece 2 onto the surface 11 to be treated can be ensured clearly and reliably. According to advantageous developments it is also the case that the initial operation or the maintenance of operation is also possible when the contact piece is located in the immediate vicinity of the skin surface as the permitted area for example a distance of up to 5 mm.

The light source 6 emits its light in the direction of the light outlet section 4. Without contact of the contact piece 2 with the surface 11 the emitted light reaches the surface 11 and is mostly scattered diffusely or reflected back and thus reaches the photodetector 9 via the light inlet section 5. As long as the contact piece 2 does not touch the surface 11 both wavelengths emitted by the light source 6 are detected by the photodetector and analyzed by the analyzing circuit 17 accordingly.

FIG. 2b shows a plan view of the application surface 3 of the contact piece 2. In the preferred configuration the light outlet section 4 has an essentially circular cross section and can also be much larger in relation to the outlet surface than the light inlet section 5. In the shown, preferred embodiment two light inlet sections 5 are provided, wherein the two light inlet sections 5 are arranged opposite one another about a center of the light outlet section 4. In a further possible configuration also additional light inlet sections can be provided as indicated in FIG. 2b by dashed circles. In this way an arrangement of light inlet sections surrounding the light outlet section 4 is formed so that also any tilting of the contact piece 2 and thus a light lateral lifting of the light outlet section 4 can be recognized.

In the shown embodiment the safety circuit 7 is integrated into the device 24 to be made safe so that the contact piece is used essentially as a spacing part. This embodiment has the particular advantage that the contact piece 2 can be configured as a disposable part. Particularly when in use in a medical or cosmetic field, sterile devices are required in order to prevent any interaction that is potentially hazardous to health of a non-sterile contact piece 2 with the treated section of the surface 11 or the tissue 12. The contact piece 2 is attached by means of a not shown assembly or locking device onto the handpiece 24, the treatment is carried out and the contact piece 2 is then disposed of. The device 24 can be configured to be impermeable to sterilization or cleaning agents, for example the area in which the emitted light radiation of the present safety device and the treatment radiation escape or enter as the reflected or scattered wavelengths can be tightly sealed by glass transparent to these wavelengths so that the device or surface can also be cleaned using aggressive cleaning agents.

A further light sensor is not shown in the Figure, which with its main detecting direction is arranged aligned in the direction of the light outlet section. By means of said light sensor all of the light reflected back into the light outlet section is detected in order to derive additional information about the quality of the application of the contact piece on the skin. In particular, in this way also a slight lifting away from full contact can be identified.

FIGS. 3 a) and b) show the principle of determining the skin contact with the present skin contact detecting device 1. A light source 6 is arranged with its main beam direction 8 in the direction of the light outlet section 4 and emits light of a first 13 and second 14 wavelength. As long as the contact piece 2, in particular the application surface 3, does not lie on the surface 11 or on the tissue 12, the light emitted from the light source 6 escapes from the light outlet section 4 and is reflected or propagated by the surface 11 or tissue 12 and thus enters the direction of the light inlet section 5. The reflected back or scattered light reaches the photodetector 9 as a first 15 and second 16 wavelength and is converted by the latter into a first 39 and second 40 signal proportional to the respective intensity.

The first 13 and second 14 emitted wavelengths are selected so that for open space propagation in air and for the reflection or propagation on the surface 11 or on the tissue 12 the damping conditions are configured such that for both wavelengths a small amount of damping is provided so that a sufficiently high intensity of the received wavelengths is ensured. The approach of the contact piece 2 to the surface 11 or the tissue is recognized by the photodetector 9 or the analyzing circuit in that
no or only very low intensity of both wavelengths is detected. In this case the contact piece 2 is removed so far from the surface or the tissue that the intensity of the reflected or scattered wavelength is in the range of the detecting threshold of the photodetector;
a uniform increase in the intensity values of both wavelengths is determined. In such a case the contact piece 2 approaches the surface 11 or the tissue, so that more parts of the wavelengths scattered or reflected on the surface 11 or the tissue enter the light inlet section 5 and are thus detected by the photodetector as an increase in intensity.

FIG. 3b shows conditions on placing the contact piece 2 on the surface 11 or on the tissue 12. On the basis of the propagation conditions for the first 13 and second 14 emitted wavelength in the tissue 12, the second emitted wavelength 14 is passed through the tissue. The first emitted wavelength 13 is damped so much by the tissue that after a very small penetration depth no more intensity can be found. This is achieved in that the first emitted wavelength 13 is selected from a range in which the tissue 12 has a high damping factor, the latter corresponds in the preferred embodiment to a wavelength in the region of less than 470 nm, in particular 450 nm. In contrast the second emitted wavelength 14 is in a range of 610 nm to 660 nm, preferably 635 nm, wherein in this frequency range comparatively less damping is provided so that incoming light is transferred and thus can reach the light inlet section 5 and thereby the detecting area 10 of the photodetector 9.

The correct application of the contact piece 2 on the surface 11 can thus be recognized in that only the second wavelength 16 is received by the photodetector 9 or in that in contrast to the intensity conditions prior to placing the contact piece 2 on the surface 11, there is a clear difference of received intensity between the first 15 and second 16 received wavelength. In contrast a slight lifting of the contact piece 2 can be recognized in that suddenly a first wavelength 15 is received again or a difference is formed of the received intensities between the first 15 and second 16 received wavelength. As soon as a non-reliable application of the contact piece 2 on the surface has been identified a non-safe operating state signal can be emitted by the analyzing circuit, after which the device to be secured, for example a laser of class 4, is immediately deactivated and thereby the persons present are not exposed to health risks.

FIG. 4 shows schematically the intensity pattern of the received wavelengths when approaching the surface up to application and then the following partial release of the contact, whereby an operating state is provided that is potentially hazardous to health which needs to be prevented in any case. To simplify the representation the intensity pattern of the two wavelengths is shown by a different type of line. The shown intensity pattern shows a possible example of an intensity pattern, in particular the principle is illustrated but the actual intensity pattern may differ from this. To simplify the representation the intensity values of the two received wavelengths have been adjusted relative to one another, the absolute intensity values will differ from one another significantly. In the situation represented it comes down to the pattern, therefore the absolute values are of secondary importance. Here too a situation is shown in which the complete application has to be ensured. According to advantageous developments also a slight lifting can be tolerated in a secure operating state.

During an approach phase 29 the intensity of the first 15 and second 16 received wavelength will increase, as because of the approach of the contact piece to the surface more light arrives from the light outlet section by reflection or propagation on the surface into the light inlet section. The second wavelength is reflected effectively by the skin so that at a greater distance the second wavelength 16 is received. The first wavelength is reflected much more weakly by the skin so that there is only a reflection with a much smaller spacing and thus a second received wavelength 15. As the approach to the skin gets closer the intensities of the two received wavelengths 15, 16 increase; it is essential in this case that both intensities increase. In the diagram this is represented by the same increase of the intensity curves.

On placing the contact piece onto the surface both wavelengths are introduced via the light outlet section into the tissue and propagate therein according to the propagation conditions. The damping that a wavelength experiences through the tissue is significant here. The time point of the application is marked in the diagram by section S1. For the first received wavelength 15 because of the strong damping through the tissue there is severe drop in the detected intensity up to complete removal, so that for the first wavelength 15 no intensity or only very little intensity can be detected by the photodetector. As the second wavelength 16 is also damped by the tissue, here too the detected intensity falls but to a much lesser extent than for the intensity of the first wavelength 15. A secure operating state 30 is thus characterized in that there is a large difference 31 between the determined intensities of the second 16 and first 15 received wavelengths or in that the intensity of the first wavelength 15 completely disappears. In the representation of the approach phase 29 shortly before contact there is no difference between the intensities of the two wavelengths. This has only been selected for illustrative purposes. As already mentioned the absolute intensity values can also differ but this will increase significantly when applying to the surface.

If the contact piece is now lifted very slightly light can come from the light outlet section directly into the light inlet section and the first wavelength 15 is detected with a noticeable intensity, whereby the difference between the first 15 and second 16 received wavelength also changes. This is the case in the diagram from state S2, which thus clearly characterizes an unsafe operating state 32. Then the non-safe operating state signal is emitted by the evaluation module and the device to be made safe can thus be deactivated in order to prevent the health of the persons involved being put at risk.

For the reliable identification of an approach of the device to skin tissue and then placing it thereon the pattern of the detected intensities is essential. In this respect according to one development the comparator circuit is designed to be state dependent, in particular in the form of a state machine. A state machine makes it possible to check for example whether the input variable is behaving according to a predefined pattern without having to specify all of the options for the pattern in detail. An approach with subsequent application can be checked by a state machine in the following manner.
The intensity of the second received wavelength increases; the intensity of the first received wavelength is below a detection threshold.
A first wavelength is detected, the intensity of which has to increase, the intensity of the detected second wavelength also has to increase further.
Having reached a maximum for both wavelengths, the intensities of both detected wavelengths fall rapidly and sharply, the intensity of the first detected wavelength disappears or is very low.
In this state the application or placing of the device on the skin is recognized.
The application is continued only until a first wavelength is detected or until an over-proportional intensity fluctuation of the second wavelength is detected.

The shown steps are only given by way of example to illustrate the principle. In particular, other steps can be provided to achieve intermediate states, for example to recognize an immediate area of the skin surface.

FIG. 5 shows a further possible embodiment, in which the breakthrough 34 of the light outlet section 4 of the contact piece 2 is connected to a device for generating a differential pressure 33. On applying the contact piece 2 onto the surface the breakthrough 34 of the light outlet section 4 in the contact piece 2 is sealed as far as possible on one side by the skin surface 11 and on the opposite side by the handpiece 24, on which the contact piece is arranged. By means of the differential pressure device 33 therefore in the breakthrough 34 there can be a reduced air pressure relative to the environment, whereby because of the largely sealed end, this differential pressure can be essentially maintained. However, it is also possible that in the region of the breakthrough 34 a defined airflow is formed so that reaction products, which are formed by the action of the optical high-energy radiation source arranged in the handpiece 24 on the surface to be processed, are transported away from the treatment area. In addition, the contact piece 2 preferably has a defined feed device 35 for incoming air. If by the differential pressure device 33 the breakthrough 34 is charged with a negative pressure, a defined amount of air flows into the breakthrough 34 via the air inlet opening 35, flows through the latter so that the removed products are removed out of the breakthrough and for example collected in a filter 36, which is arranged in the air outlet duct. To evaluate the pattern of the differential pressure the evaluation module comprises a differential pressure detecting device, by means of which it is possible to identify if the contact piece 2 has lifted slightly from the surface 11. If lifting has occurred the pressure difference produced by the differential pressure device 33 in the breakthrough will change. For example, it is also possible that the placing of the contact piece 2 on the skin surface 11 is detected by means of the previously described optically active device, afterwards the differential pressure device 33 is activated and differential pressure is formed in the breakthrough 34. This differential pressure is taken as the reference pressure, and then the application of the contact piece 2 on the surface is controlled by monitoring the differential pressure. As before a threshold can also be saved for the determined differential pressure, which is compared by the evaluation module with the currently determined difference value in order to thus prevent a systematic manipulation of the differential pressure measurement.

FIG. 6 shows a further possible embodiment of the present skin contact detecting device, in which the safety circuit 7 is integrated into the contact piece 2. The optical high energy radiation source 23 will emit its radiation 25 via a window 26 in the housing of the handpiece 24 to the outside, in particular into the light outlet section 4 of the contact piece 2. To supply the light source 6 and the analyzing circuit 17 or photodetector 9 the contact piece 2 comprises a contact section 26 facing the device 24 to be secured, which on arranging the contact piece 2 on the device 24 to be secured is connected with a contact section 27 arranged opposite in the handpiece 24. The two contact sections 26, 27 are preferably designed for transmitting electrical energy and possible electrical signals. According to one development on the application surface 3 at least one contact electrode can be arranged which is connected to the analyzing circuit 17. In this way the measurement is possible of electrical parameters of the surface or tissue, on which the contact piece 2 lies. The determination of electrical parameters by means of the contact electrode 28 and the analyzing circuit 17 provides an additional safety aspect, as by means of the electrical parameters for example also the moisture content of the tissue can be determined and thus an increase in the reliability of recognizing the application on the skin is achieved. By means of the contact electrode for example a resistance value or a capacitive or dielectrical displacement current can be determined. This development enables a further increase in the detection security of placing the contact piece 2 on the surface. The use of this development determines the application of an electrical contact on the skin, so that here if necessary separate safety requirements are used to prevent a person being put at risk by the electrical contact.

FIG. 7 shows a principle pattern of the intensity distribution saved as a reference data record for a skin type, in this case skin type III. The reference data for the remaining skin types looks similar in principle, but there are clear differences in the initial situation for the maximum value and the intensity distribution in the application. Said curves were put through a series of tests in which with a plurality of persons with different skin types, with a measuring head of the intensity pattern of the reflected wavelengths was determined during the approach to the skin. As already mentioned because of uncertainties in the skin type allocation, existing fluctuations in the skin condition (in particular tanning) and other factors dependent on the day, there may be a scattering of the detected results. As the latter are essentially random processes the distribution curve is a Gaussian distribution. Ideally the distribution function would need to have a narrow vertical line (Dirac).

The left column shows the behavior for the first wavelength, blue to UV light, the right column shows the second wavelength—red light. On the x-axis the detected intensity of the reflected wavelength is entered in standard form, the y-axis shows the distribution in standard form. Line 1 shows the situation on placing the measuring head onto the tissue, line 2 shows the situation at a spacing of 5 mm between the tissue and measuring head and line 3 shows the situation at a distance of 10 mm.

It can be seen clearly that the detected intensity of both wavelengths drops, for illustrative purposes for blue 0 mm a signal is represented, in fact the signal is very weak, almost zero. It can also be seen that the width of the distribution function decreases during the approach. It could be asserted that at a greater distance more interference can reach the photodetector and thus produce artefacts. In the Figure a spacing of 10 mm is assumed at which according to tests the maximum reflection can be established.

An approach to skin type III is now characterized in that:
At a maximum level (10 mm spacing) red is reflected strongly, with the standardized factor 0.8, the actually detected second signal will a low fluctuation margin around this average value. Blue is reflected much more weakly, the average value is about 0.6, the actually detected first signal has a much greater fluctuation margin.
Upon application the first detected signal (blue) disappears almost completely, whereas for the second detected signal (red) an even clearer signal portion is detected with an average value of 0.15.

In the reference data records such patterns are saved so that it is possible to check during the approach whether the detected pattern coincides with a saved, expected pattern. By means of the saved range details thus also statistical anomalies can be taken into account.

FIG. 8 shows a time path of the signals of FIG. 7, the second wavelength 16 (red) is complete, the first wavelength 15 (blue) is shown by a dashed line. As already mentioned during approach there is a maximum value for the reflection, then the intensities of both reflected wavelengths decrease. The intensity distribution at the maximum, in the Figure dmax, and the end intensities when placing on the skin, are characteristic for the approach to skin and in particular the skin type can be characterized thereby. During the approach firstly red light 16 with increasing intensity is detected, whereby with a large spacing signal portions are detected. With only a small spacing blue light 15 is reflected sufficiently by the skin in order to be detected. After falling below the maximum distance dmax the intensities fall, for simplicity an essentially linear drop is shown. Although the actual path can deviate from this because of the already small distance from the skin a linear approach is permissible.

The essential advantage of the present device or the present method is that by detecting an intensity pattern of two reflected wavelengths, the approach can be recognized and it can also be checked whether the approach to the skin or biological tissue has been performed. In addition it can be determined whether there has been a safe application or specific spacing ranges have been adhered to. With regard to use in the medical field it is a particular advantage that this detection can be performed contactlessly.

Lastly, it should be noted that in the variously described exemplary embodiments the same parts have been given the same reference numerals and the same component names, whereby the disclosures contained throughout the entire description can be applied to the same parts with the same reference numerals and same component names. Also details relating to position used in the description, such as e.g. top, bottom, side etc. relate to the currently described and represented figure and in case of a change in position should be adjusted to the new position. Furthermore, also individual features or combinations of features from the various exemplary embodiments shown and described can represent in themselves independent or inventive solutions.

All of the details relating to value ranges in the present description are defined such that the latter include any and all part ranges, e.g. a range of 1 to 10 means that all part ranges, starting from the lower limit of 1 to the upper limit 10 are included, i.e. the whole part range beginning with a lower limit of 1 or above and ending at an upper limit of 10 or less, e.g. 1 to 1.7, or 3.2 to 8.1 or 5.5 to 10.

The exemplary embodiments show possible embodiment variants of the skin contact detecting device, whereby it should be noted at this point that the invention is not restricted to the embodiment variants shown in particular, but rather various different combinations of the individual embodiment variants are also possible and this variability, due to the teaching on technical procedure, lies within the ability of a person skilled in the art in this technical field. Thus all conceivable embodiment variants, which are made possible by combining individual details of the embodiment variants shown and described, are also covered by the scope of protection.

FIGS. 5 and 6 show further and possibly independent embodiments of the skin contact detecting device, wherein again the same reference numbers and component names are used for the same parts as in the preceding Figures. To avoid unnecessary repetition reference is made to the detailed description in the preceding Figures.

Finally, as a point of formality, it should be noted that for a better understanding of the structure of the skin contact detecting device the latter and its components have not been represented true to scale in part and/or have been enlarged and/or reduced in size.

The underlying objective of the independent solutions according to the invention can be taken from the description.

Mainly the individual embodiments shown in FIGS. 1 to 8 can form the subject matter of independent solutions according to the invention. The objectives and solutions according to the invention relating thereto can be taken from the detailed descriptions of these figures.

List of Reference Numerals

| | |
|---|---|
| 1 | Skin contact detecting device |
| 2 | Contact piece |
| 3 | Application surface |
| 4 | Light outlet section |
| 5 | Light inlet section |
| 6 | Light source |
| 7 | Safety circuit |
| 8 | Main beam direction |
| 9 | Photodetector |
| 10 | Detecting area |
| 11 | Surface |
| 12 | Tissue |
| 13 | First emitted wavelength |
| 14 | Second emitted wavelength |
| 15 | First received wavelength |
| 16 | Second received wavelength |
| 17 | Analyzing circuit |
| 18 | Difference forming circuit |
| 19 | Comparator circuit |
| 20 | Threshold value |
| 21 | Not-safe operating state signal |
| 22 | Safe operating state signal |
| 23 | Optical high energy radiation |
| | source |
| 24 | Handpiece, device |
| 25 | Optical high energy beam |
| 26 | Contact section |
| 27 | Contact section |
| 28 | Contact electrode |
| 29 | Approach phase |
| 30 | Safe operating state |
| 31 | Difference |
| 32 | Unsafe operating state |
| 33 | Device for generating a differential |
| | pressure |
| 34 | First breakthrough |
| 35 | Air inlet opening |
| 36 | Filter |
| 37 | Second breakthrough |
| 38 | Connecting surface |
| 39 | First detected signal |
| 40 | Second detected signal |
| 41 | Reference data record for first |
| | detected signals |
| 42 | Reference data record for second |
| | detected signals |
| 43 | Database |

1-32. (canceled)

33. A method for identifying the approach and application of a device (24) onto a skin surface (11, 12), comprising a skin contact detecting device (1), which skin contact detecting device (1) comprises a contact piece (2) with an application surface (3), a light source (6) and a photodetector (9) connected to the analyzing circuit (17) wherein from the light source (6) a first (13) and second (14) wavelength is emitted via the light outlet section (4) in the direction of the skin surface (11, 12);
the first (15) and second (16) wavelength reaching the photodetector (9) via the light inlet section (5) is detected by the photodetector (9) and is converted into a first (39) and second (40) detected signal proportional to the intensity;
by the analyzing circuit a first and second time signal pattern is formed for the first (39) and second (40) detected signal;
wherein by the comparator circuit (19) of the analyzing circuit (17) the first and second signal pattern is compared with the reference data records (41, 42) saved in the database (43) of the analyzing circuit (17);
wherein in addition a safe-operating state signal (22) is emitted by the analyzing circuit (17),
if the first and second detected signal pattern coincides with reference data for first (41) and second (42) detected signal patterns.

34. The method according to claim 33, wherein the first and second signal patterns are determined by means of a smoothing function.

35. The method as claimed in claim 33, wherein the comparator circuit (19) performs a fuzzy comparison and in case of agreement emits at least one second operating state signal, in particular a warning signal.

36. The method as claimed in claim 33, wherein on the formation of the time signal patterns, a maximum value is found.

37. The method as claimed in claim 33, wherein on the formation of the time signal patterns a trend analysis is performed respectively.

38. The method as claimed in claim 37, wherein the determined trends are compared with trends saved in the reference data records (41, 42) or with reference trends determined from the reference data records.

39. The method as claimed in claim 33, wherein on placing the device (24) or the contact piece (2) onto the skin virtually no first signal (39) is determined.

40. The method as claimed in claim 36, wherein the determined signal patterns are interpolated linearly from the determined maximum value.

41. The method as claimed in claim 33, wherein the light inlet section (4) is charged with negative pressure or overpressure by a device for generating a differential pressure (33) and the differential pressure is monitored by a pressure-measuring device, and on exceeding a pressure limit value of the differential pressure the safe-operating state signal (22) is emitted and on falling below a pressure limit value of the differential pressure the not-safe operating state signal (21) is emitted.

42. A skin contact detecting device (1) for a device (24) to be secured, designed for performing a method as claimed in claim 33,
comprising
a contact piece (2) with an application surface (3),
a safety circuit (7) with a light source (6), a photodetector (9) and an analyzing circuit (17), which analyzing circuit (17) is connected to the photodetector (9),
the application surface (3) having a light outlet section (4) and a light inlet section (5) delineated therefrom,
the light source (6) having a main beam direction (8) of emitted electromagnetic radiation,
which main beam direction (8) is aligned in the direction of the light outlet section (4),
the photodetector (9) comprising a detecting area (10) for electromagnetic radiation,
which detecting area (10) is aligned in the direction of the light inlet section (5),
the light source (6) being configured for emitting light of a first (13) and at least a second (14) wavelength and the photodetector (9) is sensitive to the first (13) and second (14) wavelength and is designed for converting a received first wavelength (15) into a first detected signal (39) and the received second wavelength (16) into a second detected signal (40),
wherein
the analyzing circuit (17) comprises a database (43), in which reference data records (41, 42) are saved comprising reference data for detected first (39) and second (40) signals, the analyzing circuit being designed to form from the detected first (39) and second (40) signal a first and second signal pattern, and additionally the analyzing circuit (17) comprises a comparator circuit (19),
which comparator circuit (19) is designed for comparing the first and second signal pattern with the reference data records (41, 42) saved in the database (43) and for emitting a safe operating state signal (22).

43. The skin contact detecting device as claimed in claim 42, wherein the first wavelength (13) is less than 470 nm, in particular 450 nm.

44. The skin contact detecting device as claimed in claim 42, wherein the second wavelength (14) is in a range of 610 nm to 660 nm, in particular 635 nm.

45. The skin contact detecting device as claimed in claim 42, wherein the contact piece (2) comprises a connecting surface (38) lying opposite the contact surface (3), wherein the connecting surface (38) is designed for arranging the contact piece (2) on a device (2) to be secured.

46. The skin contact detecting device as claimed in claim 42, wherein the light outlet section (4) is connected via a first breakthrough (34) in the contact piece (2) and/or the light inlet section (5) is connected via a second breakthrough (37) in the contact piece (2) to the connecting surface (38).

47. The skin contact detecting device as claimed in claim 46, wherein the second breakthrough (37) is formed by a light conductor.

48. The skin contact detecting device as claimed in claim 42, wherein the safety circuit (7) is arranged in the device (24) to be secured.

49. The skin contact detecting device as claimed in claim 42, wherein in the light outlet section (4) a spacing device is arranged.

50. The skin contact detecting device as claimed in claim 42, wherein the light source (6) is formed by at least one light-emitting diode or a laser diode.

51. The skin contact detecting device as claimed in claim 42, wherein the light source (6) is arranged in the light outlet section (4).

52. The skin contact detecting device as claimed in claim 42, wherein in the application surface (3) at least two contact electrodes (28) are arranged which are connected to the analyzing circuit (17).

53. The skin contact detecting device as claimed in claim 42, wherein between the contact electrodes (28) and the analyzing circuit (17) a galvanically isolated signal coupler is arranged.

54. The skin contact detecting device as claimed in claim 42, wherein the light outlet section (4) and/or the light inlet section (5) is connected to a device for generating a differential pressure (33) relative to the environment.

55. The skin contact detecting device as claimed in claim 42, wherein the photodetector (9) is sensitive at least to a third wavelength.

56. The skin contact detecting device as claimed in claim 42, wherein in the detecting area (10) of the photodetector (9) or in the photodetector (9) an optical filter element is arranged.

57. The skin contact detecting device as claimed in claim 42, wherein the comparator circuit (19) is configured to be state-dependent, in particular as a state-machine.

58. The skin contact detecting device as claimed in claim 42, wherein the reference data of the reference data records (41, 42) has a hierarchy.

59. The skin contact detecting device as claimed in claim 42, wherein the reference data of the reference data records (41, 42) is saved as pairs of range specifications.

60. The skin contact detecting device as claimed in claim 42, wherein the reference data of the reference data records (41, 42) has deviation ranges.

61. The skin contact detecting device as claimed in claim 42, wherein the safety circuit (7) comprises an ambient light sensor which is connected to the analyzing circuit (17), in particular to the comparator circuit (19).

62. The skin contact detecting device as claimed in claim 42, wherein the safety circuit (7) comprises a pulse generator which is designed for controlling the light source (6) and is connected to the comparator circuit.

63. The skin contact detecting device as claimed in claim 42, wherein the safety circuit (7) comprises a further light sensor which is aligned in the direction of the light outlet section (4) and is connected to the analyzing circuit (17), in particular to the comparator circuit (19).

64. A laser device of laser class 1, comprising a laser source with a control circuit, and a skin contact detecting device, wherein the control circuit activates the laser source only on a safe operating state signal, wherein the laser source emits a radiation output greater than laser class 1, in particular class 4 radiation is emitted, and the skin contact detecting device performs a method for identifying the approach and application of a device (24) onto a skin surface (11, 12), comprising a skin contact detecting device (1), which skin contact detecting device (1) comprises a contact piece (2) with an application surface (3), a light source (6) and a photodetector (9) connected to the analyzing circuit (17) wherein from the light source (6) a first (13) and second (14) wavelength is emitted via the light outlet section (4) in the direction of the skin surface (11, 12);
the first (15) and second (16) wavelength reaching the photodetector (9) via the light inlet section (5) is detected by the photodetector (9) and is converted into a first (39) and second (40) detected signal proportional to the intensity;
by the analyzing circuit a first and second time signal pattern is formed for the first (39) and second (40) detected signal;
wherein by the comparator circuit (19) of the analyzing circuit (17) the first and second signal pattern is compared with the reference data records (41, 42) saved in the database (43) of the analyzing circuit (17);
wherein in addition a safe-operating state signal (22) is emitted by the analyzing circuit (17),
if the first and second detected signal pattern coincides with reference data for first (41) and second (42) detected signal patterns, and wherein the skin contact detecting device comprises:
a contact piece (2) with an application surface (3),
a safety circuit (7) with a light source (6), a photodetector (9) and an analyzing circuit (17), which analyzing circuit (17) is connected to the photodetector (9),
the application surface (3) having a light outlet section (4) and a light inlet section (5) delineated therefrom,
the light source (6) having a main beam direction (8) of emitted electromagnetic radiation,
which main beam direction (8) is aligned in the direction of the light outlet section (4),
the photodetector (9) comprising a detecting area (10) for electromagnetic radiation,
which detecting area (10) is aligned in the direction of the light inlet section (5),
the light source (6) being configured for emitting light of a first (13) and at least a second (14) wavelength and the photodetector (9) is sensitive to the first (13) and second (14) wavelength and is designed for converting a received first wavelength (15) into a first detected signal (39) and the received second wavelength (16) into a second detected signal (40),
wherein
the analyzing circuit (17) comprises a database (43), in which reference data records (41, 42) are saved comprising reference data for detected first (39) and second (40) signals, the analyzing circuit being designed to form from the detected first (39) and second (40) signal a first and second signal pattern, and additionally the analyzing circuit (17) comprises a comparator circuit (19),
which comparator circuit (19) is designed for comparing the first and second signal pattern with the reference data records (41, 42) saved in the database (43) and for emitting a safe operating state signal (22).
